(19) United States
(12) Patent Application Publication
(10) Pub. No.: US 2003/0170327 A1
(43) Pub. Date: Sep. 11, 2003

(54) Vitamin and zinc monomethionine compositions

(76) Inventor: Eva Marie Dahl, Sausalito, CA (US)

Correspondence Address: Gavin J. Milczarek-Desai, Durando Birdwell & Janke PLC, 2929 E. Broadway Blvd., Tucson, AZ, 85716, US

(21) Appl. No.: 10/091,611

(22) Filed: Mar. 5, 2002

(51) Int. Cl. A61K 35/78

(52) U.S. Cl. 424/736

(57) ABSTRACT

A dietary supplement composition that contains zinc monomethionine in combination with vitamin A, vitamin C, vitamin D, and a bioflavonoid that is formulated to be optimally absorbed and to help maintain normal human system functions.

### BACKGROUND

1. Field of the Invention

The invention relates in general to the field of dietary supplements and more particularly to compositions containing a combination of zinc monomethionine, vitamin A, vitamin C, vitamin D, and a bioflavonoid.

2. Description of the Related Art

The fast pace and resultant stress of modern society increasingly raises dietary health and wellness issues. Chief among these issues is that this lifestyle tends to compromise several normal functions of the healthy human immune system. One aspect of these health issues relates to vitamin and mineral intake. As processed, instant, and fast food become more the mainstay of our national diet, so too have the health concerns stemming from vitamin and mineral deficiencies. Hence, a number of vitamin and mineral supplements have been developed to address deficiencies in persons who do not receive sufficient amounts of these nutrients through their “normal” diet.

It has only recently come to be recognized that vitamin and mineral intake can have a pronounced effect on the body's general ability to protect itself against certain conditions. For instance, such “immune-system enhancing” effects for vitamin C have been well documented in clinical studies (Hemill H. Does Vitamin C Alleviate the Symptoms of the Common Cold, A Review of Current Evidence. Scand J Infect Dis 1994;26:1-6). Thus, vitamin and mineral preparations are commonly administered not only as general nutritional supplements but also to help maintain normal healthy functioning of the Body's defense systems.

The mineral zinc has come to be recognized as a substance that is essential for cell growth and proper immune functioning (Keen, Carl L. and M. Eric Gershwin. Zinc Deficiency and Immune Function. Annu. Rev. Nutr. (1990) 10:415-31). In terms of disease prevention, zinc containing lozenges, chewing gum, pills and nasal gels have been studied and shown to lessen the severity and length of infections such as the common cold. However, certain zinc supplements such as sulfate, gluconates and other similar chelates, break down too easily in the body, which results in the release of free zinc ions that can form complexes that are unusable by cells (Wedekind, K. J. et al, Methodology for Assessing Zinc Bioavailability: Efficacy Estimates for Zinc monomethionine, Zinc Sulfate and Zinc Oxide, J Anim Sci 70:178187; 1992).

Accordingly, zinc monomethionine was developed (See U.S. Pat. Nos. 3,941,818, 4,021,569 and 4,764,633). Zinc monomethionine is a 1 to 1 complex of zinc and the amino acid methionine. Studies have shown that zinc monomethionine is better absorbed, retained longer, and therefore is more effective in comparison to other forms of zinc found in nutritional supplements (Wedekind, K. J. et al, Methodology for Assessing Zinc Bioavailability: Efficacy Estimates for Zinc monomethionine, Zinc Sulfate and Zinc Oxide, J Anim Sci 70:178187; 1992).

However, the formulation and testing of a combination of zinc monomethionine and vitamins that are optimized to enhance absorption by the body and use by the immune functions has been elusive. Thus, there exists a need for a nutritional supplement that supplies the correct amounts of zinc monomethionine in combination with the proper amounts and forms of vitamins A, C and D in combination with bioflavonoids to aid the body's defenses and to maintain those systems associated with the normal healthy functioning of the immune system and with healthy mucosal membranes. SUMMARY OF THE INVENTION

The invention relates in general to a dietary supplement composition that contains zinc monomethionine in combination with vitamins A, C, and D, and bioflavonoids. Preferably the inventive supplement is provided as a set of tablets and capsules, preferably with the following amounts of each component: vitamin A about 30,000 I.U.; vitamin C about 2,000 mg; vitamin D about 1,200 IU; bioflavonoids about 500 mg; zinc monomethionine about 30 mg. Optionally, about 10 mg of rose hips may be added to increase the absorption of vitamin C.

Thus, it is a primary objective of the invention to provide a dietary supplement having a specific combination of nutrients that are optimally formulated for absorption by the body and use by cells.

Further, an object of the invention is to provide nutrients in the form of a dietary supplement that enhance the immune system's normal response to mucosal infections.

Another object of the invention is to provide a specific dietary supplement that optimally delivers the nutrients vitamin A, vitamin C, and vitamin D, in combination with bioflavonoids and zinc monomethionine, that effectively enhance the function of the immune system.

In accordance with these and other objects there is provided a nutritional supplement specifically formulated to supply the proper amounts of the vitamins A, C, D, bioflavonoids, and zinc monomethionine needed for overall general health.

Further, in accordance with the present invention, there is provided a new and improved nutritional supplement that can be conveniently used as a kit for providing the best forms of necessary nutrients, allowing the users of such a supplement to maintain their present health and positively influence their future health.

Various other purposes and advantages of the invention will 2 become clear from its description in the specification that follows. Therefore, to the accomplishment of the objectives described above, this invention includes the features hereinafter fully described in the detailed description of the preferred embodiments, and particularly pointed out in the claims. However, such description discloses only some of the various ways in which the invention may be practiced.

### DETAILED DESCRIPTION OF THE PREFERRED EMBODIMENTS

The invention relates to a dietary supplement that contains a novel combination of vitamins and minerals and that provides optimal dosages and bodily (cellular) absorption. Preferably, the invention includes about 30,000 I.U. (International Units) of vitamin A, about 2000 mg of vitamin C, about 1,200 IU of vitamin D, about 500 mg of bioflavonoids, and about 30 mg of zinc monomethionine.

The novel combination of ingredients for the invention was developed after research on potential ways to boost the immune system through dietary supplementation. The zinc supplement used in the invention is zinc monomethionine, a form of zinc that is FDA approved safe for human consumption. Zinc deficiency has been studied and evidence shows it to be responsible for a variety of immune dysfunctions. (Keen, Carl L. and M. Eric Gershwin. Zinc Deficiency and Immune Function. Annu. Rev. Nutr. (1990) 10:415-31).

A few of the major biochemical functions of zinc include the maintenance of cell membrane structure and function, as well as playing a role in skin and connective tissue metabolism and in wound healing. Surveys show that as many as one third adults in the over-50 age group have undiagnosed zinc deficiency. However, it appears that only 10% of the overall population consumes the recommended intake of zinc. Moreover, even a marginal deficiency of zinc appears to lower defenses against infection, while supplementing with low doses of zinc has been shown to improve immune function in zinc-deficient individuals (Keen, Carl L. and M. Eric Gershwin. Zinc Deficiency and Immune Function. Annu. Rev. Nutr. (1990) 10:415-31).

Vitamin A deficiencies are still fairly common worldwide and may cause many physical/psychological difficulties. Analysis of the average U.S. diet revel that only about 4,000 IU of A are provided daily. Thus, many problems associated with vitamin A deficiency, such as visual changes (e.g. night blindness) and increased infections and skin diseases, are quite common. About 90% of the storable vitamin A is done so in the liver. Storage of vitamin A is decreased in times of disease or stress and with alcohol use unless intake of Vitamin A is increased. Accordingly, chronic illness and infections can reduce the body's levels of vitamin A, thereby weakening the mucous membranes and making them more susceptible to viral infections. It is important to supplement the body's stores of vitamin A with dosages that provide new stores and are immediately usable by the mucous membranes.

Vitamin C is a water soluble vitamin that is an anti-oxidant. As an anti-oxidant, vitamin C helps prevent the free radical damage that contributes to aging and aging related symptoms. Vitamin C has been shown to stimulate the immune system (Hemill H. Does Vitamin C Alleviate the Symptoms of the Common Cold, A Review of Current Evidence. Scand J Infect Dis 1994;26:1-6). It also has been shown to decrease the production of histamines, as well as providing adrenal support, thereby reducing immediate allergy potential. Another important function of vitamin C is the formulation and maintenance of collagen. Collagen forms the basis of connective tissue found in the skin, in cartilage, and in vertebral discs joint linings. In this regard, vitamin C is needed to manufacture collagen to give support to the body, help wounds to heal, and to maintain healthy blood vessels.

Because of its roll in immunity and collagen formation, its antioxidant effect, and the adrenal support it provides, vitamin C is used to assist in treating a wide range of infections and inflammatory maladies.

Vitamin D has also been linked to immune enhancement. Interestingly, vitamin D deficiencies are becoming more common due to the use of sun screen to prevent sun exposure. As it turns out, this lack of exposure reduces the process that generates most of the body's usable vitamin D due to a lack of solar energy penetrating the skin. Since vitamin D naturally occurs in fish liver oil, this natural form is digestively well absorbed through the cell membranes.

Bioflavonoids naturally occurs with vitamin C in fruit. Bioflavonoids help in the absorption and retention of vitamin C within the body. Another main function of bioflavonoids is to increase the strength of capillaries. For example, it is known that bioflavonoid deficiency can lead to a tendency to bruise and bleed. Thus, adding bioflavonoids to the invention further optimizes the effect of vitamin C and provides support for healthy capillaries, which allow for nutrients to flow to all areas of the body including the mucosal membranes.

As would be well known in the art, pharmaceutically acceptable agents and/or carriers would be used in preparing the vitamins and/or manufacturing the tablets and capsules. For instance, the vitamin C (ascorbic acid) and zinc monomethionine of the invention are manufactured as pills (e.g. tablets and/or capsules) to provide dosages of 1,200 mg (vitamin C) and 30 mg (zinc). These pills may also contain a pharmaceutically acceptable agents and/or carriers including but not limited to; soybean oil, gelatine, maltodextrin, glycerine, cellulose, magnesium stearate, water, silicon dioxide, ethylcellulose and others known to the art.

One especially inventive aspect of the invention relates to the combination of the zinc monomethionine with vitamins A and C. Using vitamin A and zinc together augments the effects of both synergistically as zinc is needed to help release stores of vitamin A for use by the body. Furthermore when vitamin A is deficient, vitamin C seems to be lost more rapidly from the body. Thus, the invention is thought to achieve a synergistic effect among its component substances, thereby overcoming current problems relating to the absorption and utilization of useful amounts of the vitamins and minerals as described herein.

Preferably, the vitamins A and D used in the invention are provided as a standard gelatin capsule in fish liver oil form in a dosage about 30,000 IU and 1,200 IU, respectively. For vitamin A the quantity provided in fish oil is far higher than is usually the case in other forms of this supplement. One reason for this is that betacarotene is typically used in other products to supply vitamin A. Unfortunately, many people have problems (due, for instance to impaired thyroid or liver function) converting betacarotene into the usable form of vitamin A. Thus, the invention overcomes this problem and rapidly supplies an amount of vitamin A that, in combination with other vitamins and minerals, is believed to be readily absorbed and utilized by the cells of the body.

The general health effects of the invention are supported by results obtained through actual use. For example, users have provided testimonial evidence indicating that they have experienced fewer or shorter mucosal infections as a result of adding the inventive supplement to their normal dietary intake.

The invention preferably is supplied in the form of a kit. The kit contains individually packaged sets of pills that provide vitamins A, C, and D along with zinc monomethionine and bioflavonoids in the preferred dosages described above. Thus, a supplement user has each supplement ingredient of the invention in one handy packet and does not have to obtain the same individually.

Preferably, all capsules, soft gels or tablets of the invention are manufactured such that common additives, especially those that may be objectionable based on dietary restrictions or food allergies, are excluded or reduced to trace amounts. Thus, the invention preferably contains no added yeast, wheat gluten, milk or dairy additives, sodium, sugar, artificial coloring, preservatives or flavorings.

As described above, each component of the inventive supplement ideally are included in a preferred amount. However, other amounts may also be used so long as toxic dosages are avoided. For example, the range of dosages (per day) for invention could reflect the Recommended Daily Allowance (RDA), the preferred dosage, or a greater amount for each ingredient (For background on the RDA, see Recommended Dietary Allowances, 10^{th} Edition, National Academy Press, Washington, D. C. 1989). Thus, the amount of zinc monomethionine could desirably be between 10-15 mg (RDA), preferably 30 mg, and up to about 150 mg; the amount of vitamin A could desirably be between 5,000 (RDA)-10,000 IU, preferably 30,000 IU, and up to about 60,000 IU; the amount of vitamin D could desirably be between 400-1,000 IU (RDA), preferably 1,200 IU, and up to about 1,600 IU; the amount of vitamin C could desirably be between 50-60 mg (RDA), preferably 2000 mg, and up to about 10,000 mg; the amount of bioflavonoid could desirably be between 1-100 mg (no established RDA), preferably 500 mg, and up to about 1,000 mg.

Various changes in the details and components that have been described may be made by those skilled in the art within the principles and scope of the invention herein described in the specification and defined in the appended claims. Therefore, while the present invention has been shown and described herein in what is believed to be the most practical and preferred embodiments, it is recognized that departures can be made therefrom within the scope of the invention, which is not to be limited to the details disclosed herein but is to be accorded the full scope of the claims so as to embrace any and all equivalent processes and products. For example, while the preferred form for the invention is that of a pill, it is recognized that eatable or drinkable forms (e.g. snack bars or beverages) may be developed to act as delivery vehicles in lieu of pills. All references cited herein are hereby incorporated by reference in their entirety.

I claim:

1. A nutritional supplement for administration to humans, the supplement comprising:
vitamin A;
vitamin C;
vitamin D;
a bioflavonoid; and,
zinc monomethionine.

2. The nutritional supplement of claim 1, wherein the bioflavonoid is lemon bioflavonoid.

3. The nutritional supplement of claim 1, wherein vitamin A is in the form of natural fish liver oil and substantially excludes betacarotene.

4. The nutritional supplement of claim 1, wherein the vitamin D is in the form of natural fish liver oil.

5. The nutritional supplement of claim 1, further including about 10 mg of rose hips.

6. The nutritional supplement of claim 1, wherein the nutritional supplement is substantially free of yeast, wheat gluten, milk or dairy additives, corn, sodium, sugar, starch, artificial coloring, preservatives or flavoring.

7. The nutritional supplement of claim 1, further including a pharmaceutically acceptable agent or carrier selected from the group consisting of soybean oil, gelatine, maltodextrin, glycerin, cellulose, magnesium stearate, water, silicon dioxide, ethylcellulose, and combinations thereof.

8. The nutritional supplement of claim 1, wherein a dosage for each supplement component comprises: Zinc monomethionine 10 mg to about 150 mg; vitamin A 5,000 IU to about 60,000 IU; vitamin D 400 IU to about 1,600 IU; vitamin C 50 mg to about 10,000 mg; and bioflavonoid 1 mg to about 1000 mg.

9. The nutritional supplement of claim 1, wherein the supplement comprises:
about 30,000 I.U. of vitamin A;
about 2000 mg of vitamin C;
about 1,200 IU of vitamin D;
about 500 mg of bioflavonoid; and,
about 30 mg of zinc monomethionine.

10. The nutritional supplement of claim 8, wherein the bioflavonoid is lemon bioflavonoid.

11. The nutritional supplement of claim 8, wherein vitamin A is in the form of natural fish liver oil and substantially excludes betacarotene.

12. The nutritional supplement of claim 8, wherein the vitamin D is in the form of natural fish liver oil.

13. The nutritional supplement of claim 8, additionally containing about 10 mg of rose hips.

14. The nutritional supplement of claim 8, wherein the nutritional supplement is substantially free of yeast, wheat gluten, milk or dairy additives, corn, sodium, sugar, starch, artificial coloring, preservatives or flavoring.

15. The nutritional supplement of claim 8, further including a pharmaceutically acceptable agent or carrier selected from the group consisting of soybean oil, gelatine, maltodextrin, glycerin, cellulose, magnesium stearate, water, silicon dioxide, ethylcellulose, and combinations thereof.

16. A kit for providing a nutritional supplement for administration to humans, the kit comprising:
an individually packaged set of pills, wherein each set of pills provide vitamin A, vitamin C, vitamin D, a bioflavonoid, and zinc monomethionine.

17. The kit of claim 16, wherein the bioflavonoid is lemon bioflavonoid.

18. The kit of claim 16, wherein vitamin A is in the form of natural fish liver oil and substantially excludes betacarotene.

19. The kit of claim 16, wherein the vitamin D is in the form of natural fish oil.

20. The kit of claim 16, wherein the supplement additionally contains about 10 mg of rose hips.

21. The kit of claim 16, wherein the set of pills is substantially free of yeast, wheat gluten, milk or dairy additives, corn, sodium, sugar, starch, artificial coloring, preservatives or flavoring.

22. The kit of claim 16, wherein one or more pill of the set of pills further includes a pharmaceutically acceptable agent or carrier selected from the group consisting of soybean oil, gelatine, maltodextrin, glycerin, cellulose, magnesium stearate, water, silicon dioxide, ethylcellulose, and combinations thereof.

23. The kit of claim 16, wherein a dosage for each supplement component comprises: zinc monomethionine 10 mg to about 150 mg; vitamin A 5,000 IU to about 60,000 IU; vitamin D 400 IU to about 1,600 IU; vitamin C 50 mg to about 10,000 mg; and bioflavonoid 1 mg to about 1000 mg.

24. The kit claim 16, wherein the supplement comprises:
about 30,000 I.U. of vitamin A;
about 2000 mg of vitamin C;
about 1,200 IU of vitamin D;
about 500 mg of bioflavonoid; and,
about 30 mg of zinc monomethionine.
